(19) United States
(12) Patent Application Publication
(10) Pub. No.: US 2010/0075918 A1
(43) Pub. Date: Mar. 25, 2010

(54) HYALURONIDASE INHIBITORS AS ANTI-CANCER AGENTS

(76) Inventor: Vinata B. Lokeshwar, Miami, FL (US)

Correspondence Address: NIXON & VANDERHYE, PC, 901 NORTH GLEBE ROAD, 11TH FLOOR, ARLINGTON, VA, 22203, US

(21) Appl. No.: 12/516,571

(22) PCT Filed: Dec. 7, 2007
(86) PCT No.: PCT/US07/25057

§ 371 Date: May 27, 2009

Related U.S. Application Data
(60) Provisional application No. 60/873,583, filed on Dec. 8, 2006.

(51) Int. Cl. A61K 31/7016; A61P 35/04

(52) U.S. Cl. 514/53

(57) ABSTRACT

Specific inhibitors of hyaluronidase (HYA1-type hyaluronidase) are used to treat cancer, especially carcinoma and solid tumors, and/or precancerous conditions.

### CROSS-REFERENCE TO RELATED APPLICATION

This application claims the benefit of provisional Appln. No. 60/873,583, filed Dec. 8, 2007.

### FEDERALLY-SPONSORED RESEARCH OR DEVELOPMENT

The U.S. Government has certain rights in this invention as provided for by DAMD-170210005 awarded by the Department of Defense and RO1/R29-CA072821 awarded by the Department of Health and Human Services.

### FIELD OF THE INVENTION

The invention relates to hyaluronidase (HAase) inhibitors used as anti-cancer agents.

### BACKGROUND OF THE INVENTION

Hyaluronic acid is a glycosaminoglycan that promotes tumor progression and metastasis. It is made up of repeating disaccharides of D-glucuronic acid and D-N-acetylglucosamine, which are linked through alternating β-1,4 and β-1,3 glycosidic bonds.

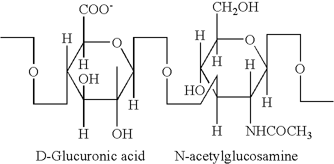

An enzyme called hyaluronidase degrades hyaluronic acid by cleaving internal β-D-N-acetylglucosaminidic linkages. But the role of hyaluronidase in cancer was largely unknown. For example, HYAL1-type hyaluronidase is an independent prognostic indicator of prostate cancer progression and a biomarker for bladder cancer. But it was controversial whether hyaluronidase (e.g., HYAL1) functions in a cancer cell as a tumor promoter or as a tumor suppressor.

In Lokeshwar et al. (2005a), a transitional cell carcinoma of the bladder cell line called HT1376 was stably transfected with HYAL1-sense (HYAL1-S), HYAL1-antisense (HYAL1-AS), or “empty” expression vectors. Whereas HYAL1-S transfected cells produced three-fold more HYAL1 than vector only transfected cells, HYAL1-AS transfected cells showed an about 90% reduction in HYAL1 production. HYAL1-AS transfected cells grew four times slower than vector only or HYAL1-S transfected cells, and were blocked in the G2-M phase of the cell cycle. HYAL1-S transfected cells were 30% to 44% more invasive, and HYAL1-AS transfected cells were about 50% less invasive than the vector only transfected cells in vitro. In xenografts, there was a 4- to 5-fold delay in the generation of palpable HYAL1-AS tumors and their weight was 9- to 17-fold less than vector only and HYAL1-S tumors, respectively (P<0.001). Whereas HYAL1-S and vector only transfected tumors infiltrated skeletal muscle and blood vessels, HYAL1-AS transfected tumors resembled benign neoplasia. HYAL1-S and vector only tumors expressed significantly higher amounts of HYAL1 (in tumor cells) and hyaluronic acid (in tumor-associated stroma) than HYAL1-AS tumors. Microvessel density in HYAL1-S tumors was 9.5- and 3.8-fold higher than that in HYAL1-AS and vector only tumors, respectively. These results show that HYAL1 expression in bladder cancer cells regulates tumor growth and progression.

In Lokeshwar et al. (2005b), prostate cancer cell lines, DU145 and PC-3 ML, were stably transfected with HYAL1-sense (HYAL1-S), HYAL1-antisense

(HYAL1-AS), or “empty” expression vectors. HYAL1-AS transfected cells were not generated for PC-3 ML because it expresses little HYAL1. HYAL1-S transfected cells produced ≦42 milliunits (moderate overproducers) or ≧80 milliunits hyaluronidase activity (high producers). And HYAL1-AS transfected cells produced <10% hyaluronidase activity when compared with vector only transfected cells (18-24 milliunits). Both blocking HYAL1 expression and high HYAL1 production resulted in a 4- to 5-fold decrease in prostate cancer cell proliferation. HYAL1-AS transfected cells were blocked in the G2-M phase of the cell cycle. High HYAL1 producers had a 3-fold increase in apoptotic activity and mitochondrial depolarization when compared with vector only transfected. Blocking HYAL1 expression inhibited tumor growth by 4- to 7-fold, whereas high HYAL1 producing transfected cells either did not form tumors (DU145) or grew 3.5-fold slower (PC-3 ML). Whereas vector only transfected cells and moderate HYAL1 producers generated. muscle and blood vessel infiltrating tumors, HYAL1-AS tumors were benign and contained smaller capillaries. Specimens of high HYAL1 producers were 99% free of tumor cells. This study shows that, depending on the concentration, HYAL1 functions as a tumor promoter or as a tumor suppressor. It provides a basis for an anti-hyaluronidase treatment for cancer.

Isoyama et al. (2006) evaluated 21 potential inhibitors of enzymatic activity against HYAL1, testicular, honeybee, and Streptomyces hyaluronidases (HAases). Among these inhibitors, polymers of poly (styrene-4-sulfonate) (i.e., MW 1400 to 990,000 or PSS 1400 to PSS 990,000) and O-sulfated hyaluronic acid analogs (sHA 2.0, 2.5, and 2.75) were the most effective. HYAL1 and bee HAases were the most sensitive, followed by testicular HAase; Streptomyces HAase was resistant to all inhibitors except PSS 990,000 and VERSA-TL 502 (i.e., PSS 10⁶ daltons). The length of the PSS polymer determined its potency (e.g., IC₅₀ for HYAL1=0.0096 μM for PSS 990,000; no inhibition for PSS 210). The presence, but not necessarily the number, of sulfonate groups on the sHA compound determined its potency (e.g., IC₅₀ for HYAL1=0.03 μM for sHA 2.0; 0.014 μM for sHA 2.75). Heparin was also a potent inhibitor of HYAL1. Other inhibitors such as gossypol, sodium aurothiomalate, 1-tetradecane sulfonic acid, and glycerrhizic acid were not effective HAase inhibitors. Hyaluronic acid alone, even at high concentration (i.e., 100 μg/mL), did not inhibit the activity of any of the four HAases. These results demonstrate that HAase inhibitors show selectivity towards inhibition of different types of HAases, which could be exploited to inhibit specific HAases involved in a variety of pathologies and other physiologic conditions. But whether a class of potential inhibitors (e.g., PSS or sHA compounds vs. HA compounds) would actually be effective against HYAL1 must be determined empirically.

Compounds to inhibit the enzymatic activity of hyaluronidase (HAase), compositions containing one or more of those compounds, and methods of treatment are taught herein to be applicable to cancer and precancerous conditions. Other advantages of the invention are discussed below or would be apparent to a person skilled in the art from that discussion.

### SUMMARY OF THE INVENTION

The invention is used as an anti-cancer agent. Thus, hyaluronidase (HAase) inhibitors may be also be used to inhibit tumor proliferation, progression through the cell cycle, or both. This may be used to manufacture a medicament (e.g., therapeutic, palliative, or prophylactic composition) to treat cancer or a precancerous condition. In particular, carcinoma or solid tumors may be treated. Typical organs of the target cell are the bladder and the prostate gland.

In a first embodiment, compounds of the invention may be used to inhibit cell proliferation (e.g., the number of cancer cells or size of tumors).

In a second embodiment, compounds of the invention may be used to inhibit progression of the cell cycle such that cancer cells are blocked at G2-M phase.

In a third embodiment, compounds of the invention may be used to reduce angiogenesis or neovascularization.

In a fourth embodiment, compounds of the invention may be used to reduce cancer cell metastasis because hyaluronidase promotes metastasis.

In a fifth embodiment, the compounds of the invention may be used to down regulate the androgen receptor (AR), and thereby provide treatment of hormone responsive and hormone refractory prostate cancers.

Compositions containing one or more compounds of the invention are also provided. Compositions may include other chemotherapeutic agents.

Further aspects of the invention will be apparent to a person skilled in the art from the detailed description and claims, and generalizations thereto.

### BRIEF DESCRIPTION OF THE DRAWINGS

FIG. 1 shows the effect of polystyrene-4-sulfonate (PSS) compounds on the growth of bladder cancer cell lines (253-J Lung or HT1376) or prostate cancer cell lines (LNCaP or DU145): 253-J Lung (FIGS. 1A to 1D), HT1376 (FIGS. 1E to 1H), LNCaP (FIGS. 1I to 1L), or DU145 (FIGS. 1M to 1P). Cancer cells cultured in RPMI 1640 medium, 10% fetal bovine serum, and gentamicin were administered various concentrations (μg/mL) of PSS compounds having different lengths (i.e., molecular weights) for up to 96 hours: PSS 990,000 (FIGS. 1A, 1E, 1I and 1M); PSS 17,000 (FIGS. 1B, 1F, 1J and 1N); PSS 6800 (FIGS. 1C, 1G, 1K and 1O); or PSS 1400 (FIGS. 1D, 1H, 1L and 1P). Cells were counted every 24 hours. Not shown are data on PC3-ML cells which express little HYAL1. PC3-ML cells were relatively resistant to growth inhibition by the PSS compounds, which suggests that the effect of PSS compounds on cell growth is mediated by HYAL1.

FIG. 2 shows the effect of sHA2.0 (FIG. 2A), sHA2.5 (FIG. 2B), or sHA2.75 (FIG. 2C) compound on growth of the bladder cancer cell line 253J-Lung. Data are shown as mean ±SD.

FIG. 3 shows the effect of various concentrations (μg/mL) of sHA2.0 (FIG. 3A), sHA2.5 (FIG. 3B), or sHA2.75 (FIG. 3C) compound on growth of the bladder cancer cell line HT1376. Data are shown as mean ±SD.

FIG. 4 shows the effect of various concentrations (μg/mL) of sHA2.75 compound on growth of LNCaP (FIG. 4A) and DU145, MATLyLu, or PC3-ML (FIG. 4B) after 96 hours. Data are shown as mean ±SD.

FIG. 5 shows the effect of sHA15k or sHA8k treatment on the growth of prostate cancer cells. LNCaP, DU145, MATLyLu, or PC3-ML were exposed to various concentrations (μg/mL) of sHA15k compound (FIG. 5A) or sHA8k compound (FIG. 5B) in culture medium, and then their cell numbers were counted after 96 hours. Data are shown as mean ±SD.

FIG. 6 shows the effect of sHA2k, sHA8k, or sHA15k treatment on growth of bladder cancer cells. 253J-Lung was exposed to various concentrations (μg/mL) of sHA2k, sHA8k, or sHA15k for 96 hours in culture medium, and then their cell numbers were counted. Data are shown as mean ±SD.

FIG. 7 shows the effects in bladder cancer cells of sHA2.75 treatment on G2-M regulators (cyclin B1, cdk1, cdc25c, Chk1, Chk2, Erk1/2, c-jun, JNK1, JNK2/3, and wee1). 253J-Lung was exposed to various concentrations (μg/mL) of sHA2.75 (FIG. 7A) or vector (V) or HYAL1-sense (HYA-S) stable transfectants of RT4 (FIG. 7B), and G2-M regulators were then analyzed by immunoblotting. Actin served as a loading standard for immunoblotting.

FIG. 8 shows the effect in prostate cancer cells of sHA2.75 treatment on androgen receptor (AR) levels. LNCaP was exposed to various concentrations (μg/mL) of sHA2.75 compound, and AR was analyzed by immunoblotting. Actin served as a loading standard for immunoblotting.

FIG. 9 shows the effect in prostate cancer cells of sHA2.75 treatment on prostate-specific antigen (PSA) levels. LNCaP was exposed to various concentrations (μg/mL) of sHA2.75 compound, and PSA (ng/mL per 10⁵ cells) in conditioned medium (CM) was analyzed by ELISA after 48 hours. The inset shows immunoblot analysis of PSA (h-PSA: human PSA control).

FIG. 10 shows the effect of sHA2.75 treatment on weight of mice.

FIG. 11 shows the effects of sHA2.75 treatment on the latency and growth of bladder cancer tumors, which were generated by subcutaneous implant in mice of the 253J-Lung cancer cells.

FIG. 12 shows sHA2.75 levels in mouse plasma as measured by carbazole assay (total GAG levels) following intraperitoneal (i.p.) or oral administration. Triplicate measurements with two mice per point.

### DETAILED DESCRIPTION OF SPECIFIC EMBODIMENTS

Concentrations of hyaluronic acid (HA) are elevated in cancer tissues, where it most likely promotes tumor metastasis. An elevated urinary HA level was shown to serve as an accurate marker for detecting bladder cancer regardless of the tumor grade. High molecular mass HA (>10⁵ daltons) is anti-angiogenic whereas small fragments of HA (3-25 disaccharide units) are angiogenic. HA is the only non-sulfated glycosaminoglycan, all others are sulfated. Hyaluronidase (HAase) inhibitors are shown to provide useful anti-cancer agents.

In humans, six HAase genes cluster into two tightly-linked triplet gene families on chromosome 3p21.3 (HYAL1, HYAL2, and HYAL3) and chromosome 7q31.3 (HYAL4, HYALP1, and PH20). HYAL1-type HAase is present in human serum and urine. HYAL1 was shown to be the major tumor-derived HAase expressed in bladder, head and neck, and prostate cancer cells. Elevated HYAL1 levels serve as an accurate marker for detecting intermediate- and high-grade bladder cancer and as a prognostic indicator for prostate cancer. HYAL1-type HAase also promotes bladder tumor growth, muscle invasion, and angiogenesis. Therefore, HYAL1-type HAase inhibitors provide useful anti-cancer agents.

Many synthetic and naturally occurring compounds act as HAase inhibitors: high molecular weight poly(styrene-4-sulfonate) (PSS), sodium aurothiomalate, fatty acids, fenoprofen, glycerrhizic acid, gossypol, heparin, plant derived compounds, and O-sulfated HA (sHA) oligosaccharides. The smaller sHA oligosaccharides have better solubility, greater bioavailability, and less toxicity than previously studied inhibitors of HAase and provide a new class of anti-cancer agents.

For example, from 1 to 25,000 (preferably at least 3, at least 5, at least 10, at least 100, at least 1000, at least 10,000, at most 5, at most 10, at most 30, at most 100, at most 1000, or at most 10,000) repeats of a disaccharide unit illustrated in Formula I may be used in the invention. Modification of the compound by sulfation at positions 1, 2, 3 or any combination thereof is preferred.

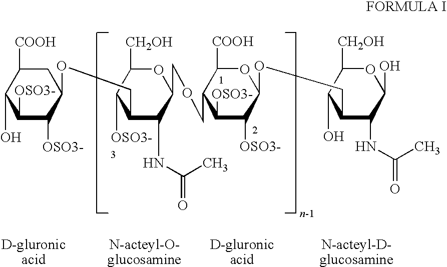

Each disaccharide unit may be modified by sulfation at two or three positions. For example, at least 25% or 50% of the disaccharide units may contain two sulfates and at least 25% or 50% of the disaccharide units may contain three sulfates.

Compounds may be used as a medicament or to formulate a composition with one or more of the utilities disclosed herein. A “pharmaceutical” composition further contains a physiologically-acceptable vehicle (e.g., buffered saline, water for injection), and is produced and stored under aseptic conditions (e.g., bacterial contamination of less than 10 cfu/100 mL). The composition may further comprise components useful for delivering compound(s) to its site of action. The choice and addition of such vehicles and carriers to the composition are within the level of skill in the art. Compounds or compositions may be administered in vitro to cells in culture, in vivo to cells in the body, or ex vivo to cells outside of the subject that may later be returned to the body of the same subject or another. The subject is a human in need of treatment (e.g., a patient affected by cancer and its metastasis, or at risk for its development) or an animal model for cancer and its pathogenesis.

The cancer may be a carcinoma or solid tumor. Precancerous conditions may also be treated by preventing the development of cancer. Typical organs in which the cancer may develop include breast, bladder, colon, head and neck, intestine, kidney, prostate gland, rectum, stomach, and upper urinary tract. Cancer may then metastasize to lymph node, lung, bone, liver, and brain but it may appear almost anywhere. Treatment for metastatic cancer varies according to the site of spread, prior treatments, and health of the subject. Commonly used to visualize solid tumors and metastases are bone scans, CT scans, MRI scans, PET scans, sonograms, and X-rays. Physical exams and laboratory screening tests may initially indicate the possibility of cancer, and this may be confirmed by obtaining a surgical biopsy and processing the biopsy for histopathology.

Pharmaceutical compositions may be administered as a formulation adapted for delivery to the systemic circulation or locally to a solid tumor in vivo. Alternatively, pharmaceutical compositions may be administered to culture medium in vitro. In addition to the active compound, such compositions may contain physiologically-acceptable vehicles, carriers, and other ingredients known to facilitate administration and/or enhance uptake (e.g., dimethyl sulfoxide, lipids, polymers, affinity-based cell specific-targeting systems). The composition may be incorporated in a gel, sponge, or other permeable matrix (e.g., formed as pellets or a disk) and placed in proximity to the tumor for sustained, local release. It may be administered in a single dose or in multiple doses which are administered at different times.

Pharmaceutical compositions may be administered by any known route. By way of example, the composition may be administered by a mucosal, pulmonary, topical, or other localized or systemic route (e.g., enteral and parenteral). The term “parenteral” includes subcutaneous, intradermal, intramuscular, intravenous, intraarterial, intrathecal, intravesical, and other injection or infusion techniques, without limitation. Oral administration is preferred.

Suitable choices in amounts and timing of doses, formulation, and routes of administration can be made with the goals of achieving a favorable response in the subject with cancer or at risk thereof because of a precancerous condition (i.e., efficacy), and avoiding undue toxicity or other harm thereto (i.e., safety). Therefore, “effective” refers to such choices that involve routine manipulation of conditions to achieve a desired effect. In particular, an effective amount of the agent(s) is present to inhibit hyaluronidase activity in cells at a particular site or metastasized to other sites. For an effective amount of inhibitor to be used to treat a subject with cancer or at risk thereof, reduction of at least 50% of HAase activity, at least 75% of HAase activity, at least 90% of HAase activity, or at least 95% of HAase activity may be preferred.

In a first embodiment, an effective amount of inhibitor may inhibit cell proliferation (e.g., the number of cancer cells or size of tumors) in vivo or in vitro by at least 90% or at least 95%.

In a second embodiment, an effective amount of inhibitor may inhibit progression of the cell cycle in vivo or in vitro such that at least 90% or at least 95% cancer cells are blocked at G2-M phase.

In a third embodiment, an effective amount of inhibitor may reduce angiogenesis or neovascularization by at least 90% or at least 95%.

In a fourth embodiment, an effective amount of inhibitor may reduce cancer cell metastasis by at least 90% or at least 95%.

A bolus administered over a short time once a day is a convenient dosing schedule. Alternatively, the effective daily dose may be divided into multiple doses for purposes of administration, for example, two to six doses per day. Dosage levels of active ingredients in a pharmaceutical composition can also be varied so as to achieve a transient or sustained concentration of the compound in a subject, and to result in the desired therapeutic, palliative, or prophylactic response. But it is also within the skill of the art to start doses at levels lower than required to achieve the desired therapeutic, palliative, or prophylactic effect and to gradually increase the dosage until that effect is achieved.

The amount of compound administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration; and the like. For systemic administration, passage of the compound or its metabolite through the circulation to the tumor cells should not be critical. It should be understood that the specific dose level to be achieved for any particular subject may depend on a variety of factors, including age, gender, health, medical history, weight, combination with one or more other agents, and severity of disease.

The term “treatment” of cancer refers to, inter alia, reducing or alleviating one or more symptoms in a subject, preventing one or more symptoms from worsening or progressing, promoting recovery or improving prognosis, and/or preventing disease in a subject who is free therefrom as well as slowing or reducing progression of existing disease. For a given subject, improvement in a symptom, its worsening, regression, or progression may be determined by objective or subjective measure. Efficacy of treatment may be measured as an improvement in morbidity or mortality. Palliative (e.g., improving quality of life) or preventative (e.g., preventing development of disease or the incidence of relapse) methods are also considered treatment. Neo-adjuvant therapy to shrink a primary tumor and to make local therapy (e.g., surgery or radiation therapy) more effective and adjuvant therapy to reduce recurrence and/or the chance of resistance developing are also considered treatment.

In combination treatment, chemotherapy and/or cryotherapy and/or hormone therapy and/or radiation therapy (e.g., brachytherapy or teletherapy) and/or surgery may be used together with administration of the compound(s). Known anti-cancer agents that may be administered with the compound(s) to treat bladder cancer include docetaxel, gallium nitrate, gemcitabine, ifosfamide, paclitaxel, trastuzumab, or any combination thereof. Known anti-cancer agents that may be administered with the one or more compounds to treat prostate cancer include bicalutamide, docetaxel, estramustine, flutamide, goserelin, leuprolide, mitoxantrone, paclitaxel, prednisone, trastuzumab, or any combination thereof. Other known anti-cancer agents include cetuximab, erlotinib, etoposide, irinotecan, and topotecan. The one or more compounds may be administered before and/or during and/or after the known anti-cancer agent(s).

The amount which is administered to a subject is preferably an amount that does not induce toxic effects which outweigh the advantages which result from its administration. Further objectives are to reduce in number, diminish in severity, and/or otherwise relieve suffering from the symptoms of the disease as compared to recognized standards of care.

Production of compounds according to present regulations will be regulated for good laboratory practices (GLP) and good manufacturing practices (GMP) by regulatory agencies (e.g., U.S. Food and Drug Administration or European Medicines Agency). This requires accurate and complete recordkeeping, as well as monitoring of QA/QC. Oversight of patient protocols by agencies and institutional panels is also envisioned to ensure that informed consent is obtained; safety, bio-activity, appropriate dosage, and efficacy of products are studied in phases; results are statistically significant; and ethical guidelines are followed. Similar over-sight of protocols using animal models, as well as the use of toxic chemicals, and compliance with regulations is required.

The following examples further illustrate specific embodiments of the invention and are not intended to limit its scope, which is described above.

### EXAMPLES

### Materials & Methods

Gossypol, fenoprofen, 1-tetradecane sulfonic acid, glycerrhizic acid, and heparin were purchased from Sigma Chemicals (St. Louis, Mo.). Sodium salts of poly(styrene-4-sulfonates) of an average molecular weight 210 (PSS 210); 1400 (PSS 1400); 4300 (PSS 4300); 6800 (PSS 6800); 17,000 (PSS 17,000); 32,000 (PSS 32,000); 49,000 (PSS 49,000); 77,000 (PSS 77,000); 150,000 (PSS 150,000); 350,000 (PSS 350,000); and 990,000 (PSS 990,000) were purchased from Fluka Biochemical (Buchs, Switzerland). VERSA-TL 502, a poly(styrene-4-sulfonate) of average molecular weight 1×10⁶ daltons was provided by Alco Chemicals (Chattanooga, Tenn.).

HYAL1 was partially purified from the conditioned medium of HT1376 bladder cancer cells stably transfected with HYAL1 cDNA encoding the full-length protein inserted into an expression vector (Lokeshwar et al., 2005a). Briefly, a stable HYAL1-expressing clone having about 40 mU/mL activity was expanded in T75 cm² flasks. At about 60% confluence, cultures were washed three times in phosphate buffered saline and incubated in RPMI 1640 medium containing transferrin, and selenium. Following a 48 hour incubation, the conditioned medium was collected and HYAL1 was partially purified as described by Lokeshwar et al. (1999).

sHA derivatives with different degrees of sulfation (i.e., O-sulfated groups) were synthesized as described by Barbucci et al. (1995) and Isoyama et al. (2006). The amount of SO₃⁻ pyridine determines the number of O-sulfated groups that are added on the HA polymer. For example, the tributylammonium salt of HA and SO₃⁻ pyridine were mixed at a ratio of 1:4, 1:8.4 (to 1:10), or 1:15 to yield sHA2.0 (2k), sHA2.5, or sHA2.75 respectively. The number of sulfate groups present in each sHA compound was determined by elemental sulfur and nitrogen analyses (Mikroanalytisches Labor Pascher, Remagen-Bandorf, Germany). sHA2.0 is a sulfated HA polymer in which 100% of the disaccharide units present in the HA polymer have two sulfate groups, whereas, in sHA2.5, 50% of disaccharide units contain two sulfate groups and the remaining 50% contain three sulfate groups. sHA2.75 is a sulfated HA polymer in which 75% of the disaccharide units contain three sulfate groups and the remaining 25% contain two sulfate groups (Formula I).

The three positions at which sulfation can occur is illustrated above as 1, 2, and 3. Depending upon whether it is sHA2.0, sHA2.5, or sHA2.75, the number of disaccharide units with two or three sulfate groups will be different.

HPLC-purified preparations of HA oligosaccharides with an average molecular weight of about 15 kD (˜22 disaccharide repeats), about 8 kD (˜13 disaccharide repeats), and about 2 kD (˜3 disaccharide repeats) were obtained from Genzyme Corp. The sizes of the HA oligosaccharides were chosen to represent a broad range of HA oligosaccharides that are generated when HAase degrades HA. For example, the 8 kD oligosaccharides represents an angiogenic fragment (Lokeshwar et al., 1996), 15 kD is possibly nonangiogenic, and the 2 kD oligosaccharide represents the smallest HA oligosaccharide that can be generated by HAase degradation. These HA oligos were converted to tributylammonium salt and sulfated using SO₃⁻ pyridine at a ratio of 1:15. Elemental sulfur and nitrogen analyses showed that like sHA2.75, 75% of disaccharide units in sHA15k, sHA8k, and sHA2k contain three sulfate groups and the remaining 25% contain two sulfate groups.

Since sHA2.75 and small sHA oligos containing the same number of repeating sulfated disaccharide units, the number of sulfated disaccharide units at the same concentration of a sHA compound (e.g., 1 mg/mL solution of either sHA2.75 or sHA-oligo) is identical, i.e., it is independent of the polymer length and the average molecular weight of the polymer (Isoyama et al., 2005).

### Effect of HAase Inhibitors on the HAase Activity

HAase ELISA-like assay was performed as described earlier by Stern & Stern (1992), Lokeshwar et al. (2000), and Lokeshwar et al. (2001). A microtiter well plate coated with human umbilical cord HA (MP Biomedicals; Irvine, Calif.) was incubated with about 40 mU/mL of HYAL1-type HAase in the presence or absence of various HAase inhibitors in assay buffer. For the HYAL1-type activity assay, 0.1 M sodium formate, 0.15 M NaCl, and 0.02% bovine serum albumin buffer (pH 4.2) was used as the assay buffer.

The effect of HAase inhibitors on the activity of HYAL1-type HAase was tested at 0, 1, 2, 4, 6, 8, 10, 20, 40, 60, 80 and 100 μg/mL concentrations of each inhibitor. The plate was incubated at 37° C. for 16 hours. Following incubation, the degraded HA was washed off and the HA remaining on the microtiter plate was determined using a biotinylated HA-binding protein and an avidin-biotin detection system (Vector Laboratories, Burlingame, Calif.) according to Pham et al. (1997). Each plate was developed for 3 min and the reaction was terminated by adding 3 N HCl. HAase activity (mU/mL) was calculated from a standard graph prepared by plotting known amounts of Streptomyces HAase (10⁻⁴ U/mL) versus (Control (no enzyme) OD_{405 nm}-Sample OD_{405 nm}). IC₅₀ for each inhibitor was calculated as the concentration (μg/ml) of an inhibitor required to inhibit 50% of the activity of the HAase tested; 100% activity was defined as enzyme activity obtained in the absence of any inhibitor. The data presented are mean ±SEM from duplicate measurements in two independent experiments.

Microtiter plates were coated with 100 μL of 0, 0.5, 1, 2.5, 5, 7.5 and 10 μg/mL of human umbilical cord HA at 37° C. for 4 hours. Following incubation, each well received about 20 mU/mL of HYAL1-type HAase and 0, 6, 10 or 20 μg/mL of an inhibitor, in a total volume of 100 μL of the assay buffer described above. Control wells received inhibitor at a particular concentration but no enzyme. The plate was incubated at 37° C. for 16 hours and then developed as described above. The velocity was defined as the amount of product formed during incubation. The amount of product (i.e., HA degraded) was calculated by subtracting the amount of HA remaining on the well from the amount of HA that was used to coat the wells. The amount of HA remaining in a well was calculated from OD_{405 nm} in control wells (i.e., wells containing no enzyme) and OD_{405 nm} in sample wells (i.e., wells containing enzyme and inhibitor). Kₘ, Vₘₐₓ, and Ki (for both competitive and uncompetitive inhibition) were calculated by plotting Lineweaver-Burk double reciprocal plots (1/μM product versus 1/μM substrate). These values were expressed as μM by assuming the molecular mass of HA to be 500,000 daltons. The molecular mass of HA was determined by gel filtration chromatography performed on a Sepharose S-300 column. The graphs were plotted using the GraphPad Prism Software Program (version 3.1) and Ki value for each inhibitor was calculated from the extrapolated X- and Y-intercepts and slope. Ki for the competitive inhibition component of mixed inhibition was calculated from the equation: α=1+1/Ki where α=1/Vₘₐₓ. The Ki for the uncompetitive inhibition component of mixed inhibition was calculated as, α′=1+1/Ki where α′=Y-intercept×Vₘₐₓ. The Ki values were expressed as concentration (μM) of the inhibitor, and were calculated from the molecular mass of the inhibitor.

TABLE 1
Potential Inhibitors of HYAL1 Activity
(N.D. = no inhibition detected)

| HYAL1 Inhibitor | IC₅₀ (μg/mL) |
|---|---|
| PSS 990,000 | 9.6 |
| PSS 350,000 | 13.15 |
| PSS 150,000 | 10.5 |
| PSS 77,000 | 14.8 |
| PSS 49,000 | 15.2 |
| PSS 32,000 | 13.5 |
| PSS 17,000 | 15.2 |
| PSS 6800 | 22.71 |
| PSS 1400 | 26.8 |
| PSS 4300 | 8.48 |
| PSS 210 | N.D. |
| sHA2.0 | 15 |
| sHA2.5 | 13.9 |
| sHA2.75 | 6.8 |
| sHA15k | 8.3 |
| sHA8k | 7.5 |
| sHA2k | 7.1 |
| Heparin | 15.1 |
| Gossypol | N.D. |
| Fenoprofen | N.D. |
| 1-Tetradecane | 18.9 |
| sulfonic acid | |
| Glycerrhizic acid | 32.4 |

As shown in Table 1, compounds inhibited HYAL1 activity with IC₅₀ ranging between 10 and 20 μg/mL. Furthermore, the IC₅₀ of sHA2.75, sHA15k, sHA8k, and sHA2k oligosaccharides is similar. Therefore, the sHA compounds are inhibitors of HYAL1 regardless of polymer length because the amount of sulfated disaccharide units at a given weight concentration of a sHA compound (e.g., 1 mg/mL solution of either sHA2.75 or sHA oligo) is the same. But not all compounds are effective inhibitors of HYAL1 activity.

### PSS Compounds Inhibit Cancer Cell Proliferation

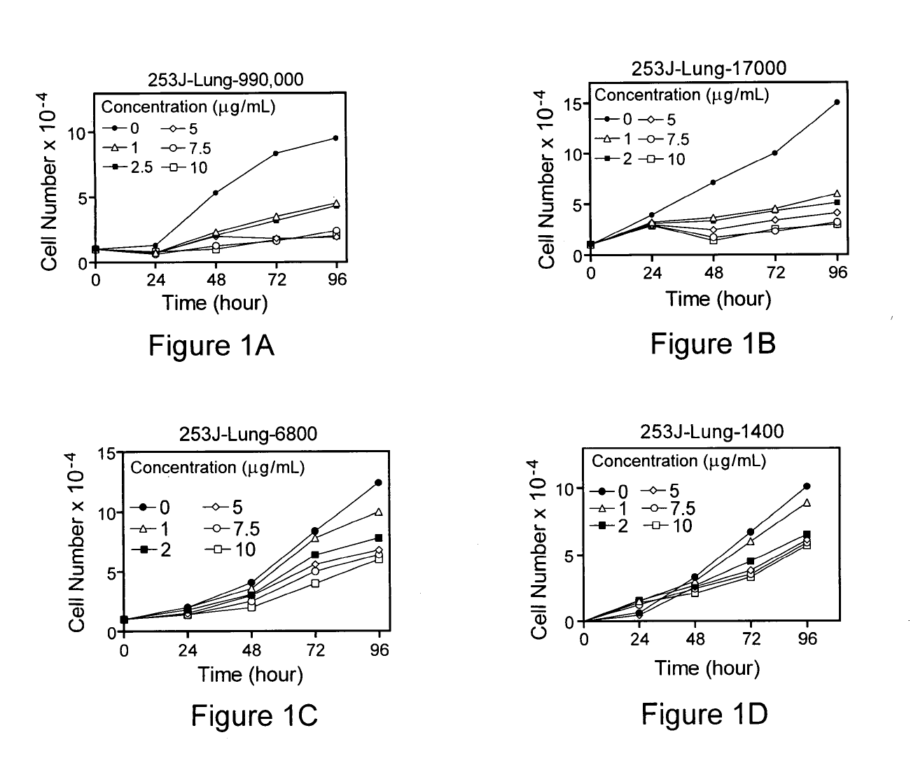

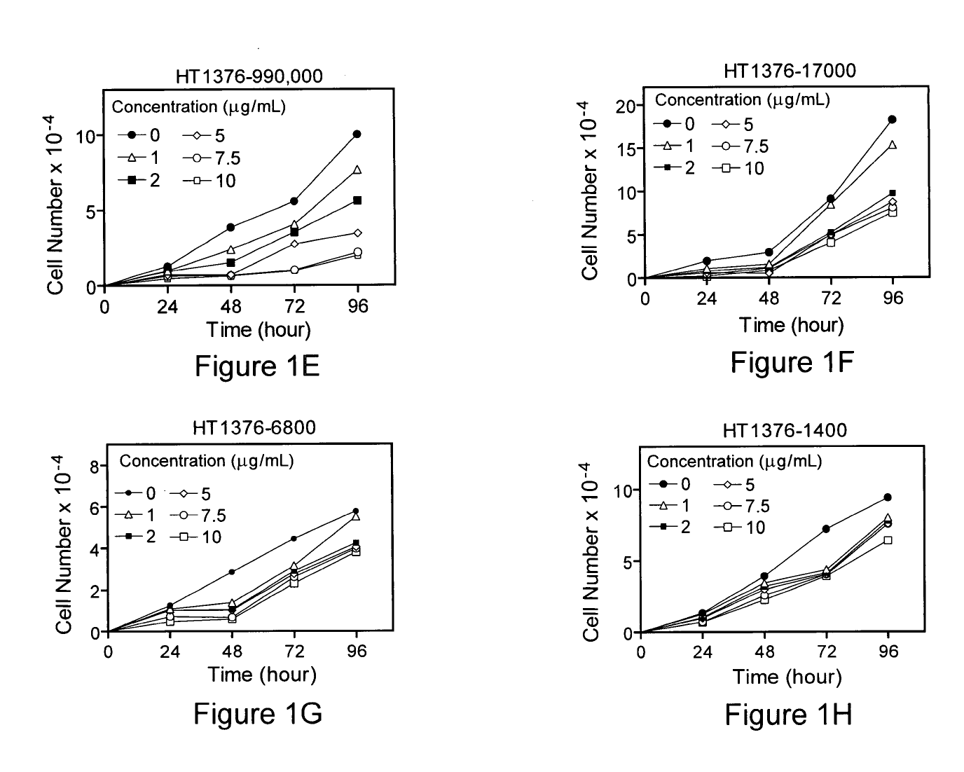

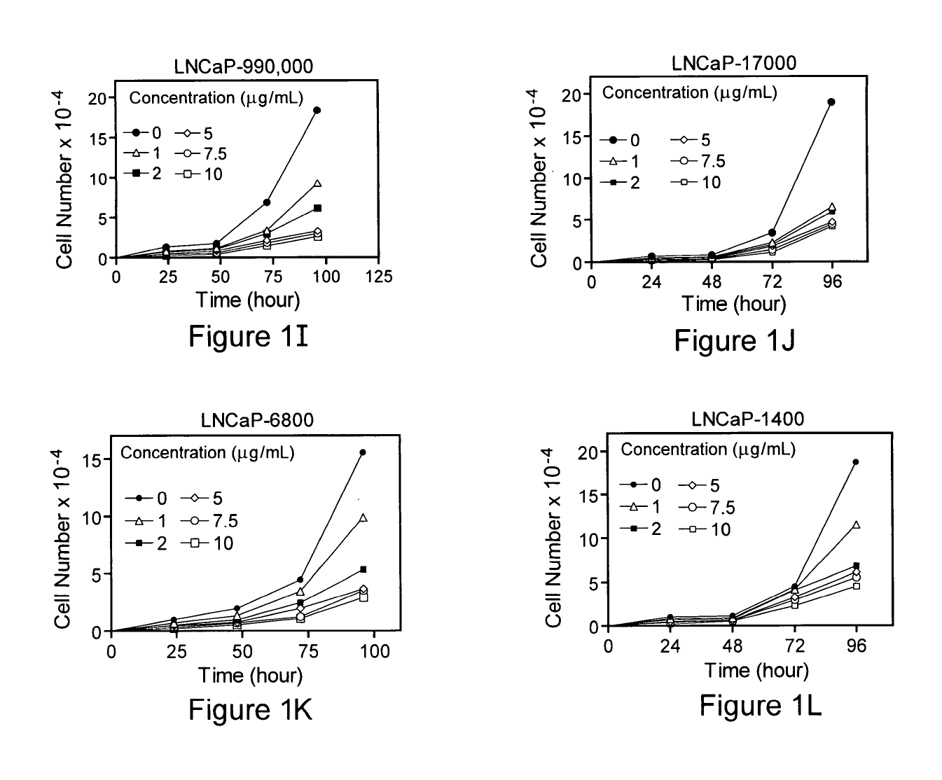

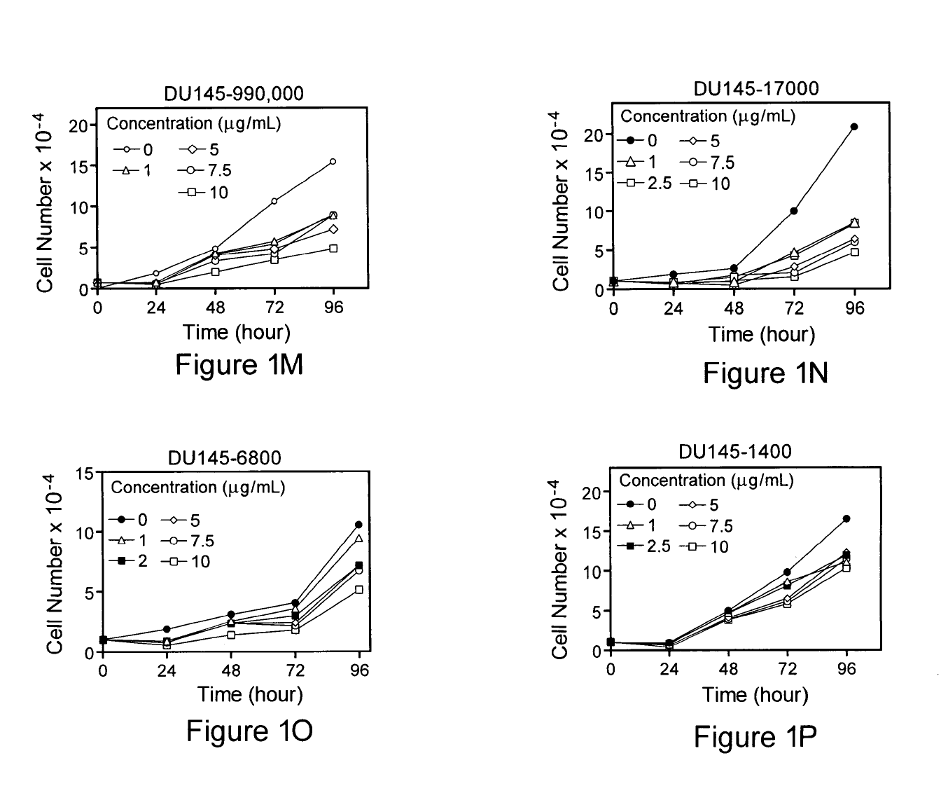

Bladder cancer cells (HT1376, 253 J-Lung), androgen-dependent prostate cancer cells (LNCaP), and androgen-independent prostate cancer cells (DU145, PC3-ML) were used. Bladder and prostate cancer cells (2×10⁴ cells/well) were plated on 24-well culture plates in RPMI 1640 medium, 10% fetal bovine serum, and gentamicin. They were treated at various concentrations of PSS 999,000; PSS 17,000; PSS 6800; and PSS 1400. Cells were incubated at 37° C. in a CO₂ incubator and cell numbers were counted from duplicate wells every 24 hours. As shown in FIG. 1, all of the PSS compounds inhibited the growth of both bladder cancer cells (FIG. 1A) and prostate cancer cells (FIG. 1B). While PSS 999,000 and PSS 17,000 compounds were equally effective in inhibiting the growth of bladder and prostate cancer cells (ED₅₀ of about 5 μg/mL), shorter polymers had lower efficacy in inhibiting the growth of cancer cells. For example, PSS 1400 was the least effective in inhibiting the growth of all cancer cells (ED₅₀ greater than about 10 μg/mL). It is noteworthy that the IC₅₀ of PSS 6800 and PSS 1400 to inhibit HYAL1 activity is 2.5-fold higher than that for PSS 999,000 and PSS 17,000. This suggests that the cell proliferation inhibitory activity of PSS compounds is related to their HYAL1 inhibitory activity.

### sHA Compounds Inhibit Cancer Cell Proliferation

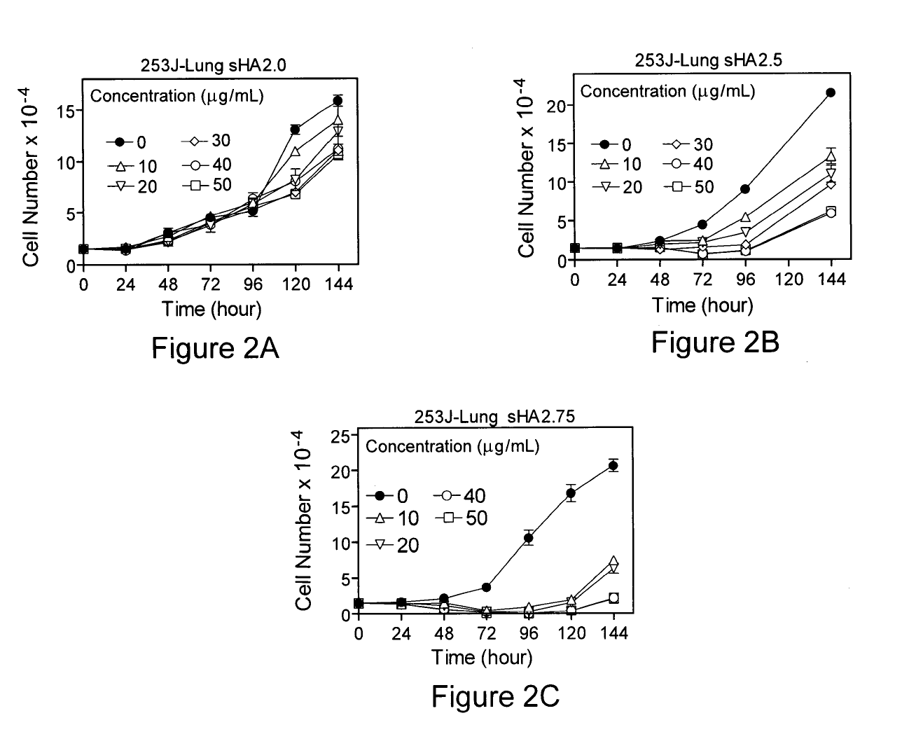

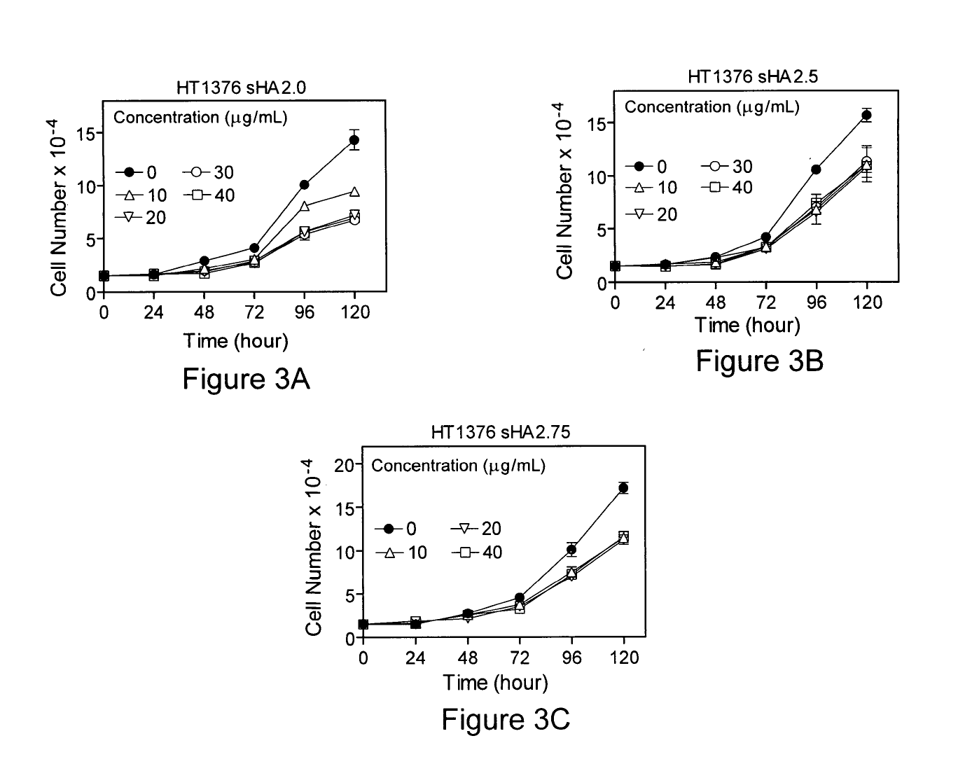

Bladder cancer cells (2×10⁴ cells/well) were plated on 24-well culture plates in RPMI 1640 medium, 10% fetal bovine serum, and gentamicin then treated with various concentrations of sHA2.0, sHA2.5, and sHA2.75 for 120 hours. Cells were incubated at 37° C. in a CO₂ incubator and cell numbers were counted from duplicate wells every 24 hours. As shown in FIG. 2, there was a correlation between the degree of sulfation in sHA compounds and their ability to inhibit 253J-Lung bladder cancer cell proliferation. For example, the ED₅₀ of sHA2.0 to inhibit cell growth is about 40 μg/mL whereas the ED₅₀ of sHA2.75 is about 10 μg/mL. For HT1376 bladder cancer cells, all three sHA compounds were effective with ED₅₀ of about 30 μg/mL (FIG. 3). Treatment of cells with HA even up to 100 μg/mL had no inhibitory effect on the growth of tumor cells.

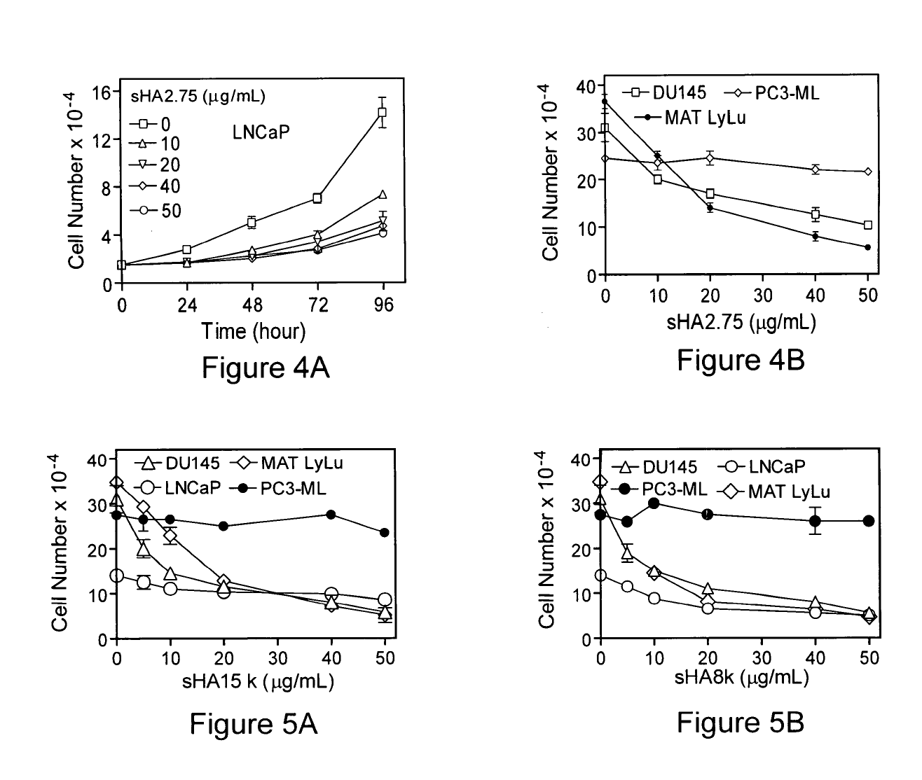

The effect of sHA2.75 treatment on the proliferation of prostate cancer cells was also studied using the proliferation assay as described above. FIG. 4A shows the effect of sHA2.75 on the growth of LNCaP cells over a period of 96 hours with ED₅₀ of about 10 μg/mL. FIG. 4B shows the effect of sHA2.75 on the growth of DU145, PC3-ML, or Dunning rat MAT LyLu cells. For DU145 and MAT LyLu cells, the ED₅₀ of sHA2.75 is about 20 μg/mL. sHA2.75 did not inhibit PC3-ML growth even at concentrations greater than 50 μg/mL, and since PC3-ML cells do not express HYAL1, the growth inhibitory effect of sHA2.75 on prostate cancer cells appears to be due to HYAL1 inhibition.

### sHA Oligosaccharides Inhibit Cancer Cell Proliferation

Prostate cancer cells (LNCaP, DU145, and MAT LyLu) were cultured in 24-well plates in medium and treated with either sHA15k or sHA8k (0, 5, 10, 20, 40, and 50 μg/mL) for 96 hours. Following incubation, their cell numbers were counted in duplicate wells. As shown in FIG. 5, sHA8k was more effective in inhibiting LNCaP cells (ED₅₀ of about 15 μg/mL) and MAT LyLu cells (ED₅₀ of about 10 μg/mL) than sHA15k. But DU145 cells were equally sensitive to sHA8k and sHA15k (ED₅₀ of about 10 μg/mL for both). PC3-ML cells were resistant to growth inhibition by both compounds, which confirms that the growth inhibitory effect of sHA oligos is mediated by inhibition of HYAL1 activity.

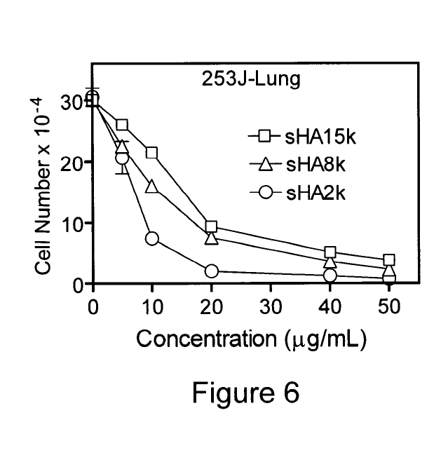

To study the effect of sHA oligosaccharides on tumor cell growth, 253J-Lung bladder cancer cells were plated in 24-well plates in culture medium and then exposed to various concentrations of sHA15k, sHA8, and sHA2k. Following incubation, cell numbers were counted. As shown in FIG. 6, the ED₅₀ for sHA2k is about 7 μg/mL and for sHA15k and sHA8k it is about 10 μg/mL. These results show that both sHA2.75 and sHA oligosaccharides with 2.75 degree of sulfation are equally effective in inhibiting the growth of cancer cells. Furthermore, the growth inhibition occurs regardless of the length of the polymer. Therefore, further studies were conducted using sHA2.75 and/or sHA2k as examples of the sHA compounds.

sHA2.75 Inhibits Cell Cycle Progression

253J-Lung bladder cancer cells and LNCaP prostate cancer cells were exposed to sHA2.75 (0 to 40 μg/mL) for 72 hours. Following incubation, the cells were lysed in hypotonic solution containing a propidium iodide (0.1% sodium citrate, 0.4% NP40 detergent, and 25 μg/mL propidium iodide) and analyzed in an EPICS XL flow cytometer equipped with a long pass red filter FL3 (630 nm). FL3 histograms were analyzed for estimating cell cycle phase distribution using the Modift Easy (Lite) program (Veritas Software, Mountain View, Calif.; Lokeshwar et al., 2005a, 2005c). All samples were assayed in duplicate in two independent experiments. As shown in Table 2, sHA2.75 at μg/ml arrests cells in the G2-M phase of the cell cycle, which suggests that sHA2.75 inhibits cell proliferation by blocking cells in the G2-M phase of the cell cycle. HYAL1-antisense transfectants were also blocked in G2-M phase.

TABLE 2
Cell cycle analysis of cancer cells

| Cell line | Control | sHA2.75 |
|---|---|---|
| LNCaP | G0-G1: 47.9% | G0-G1: 48.9% |
| | S: 39.2% | S: 22.6% |
| | G2-M: 12.9% | G2-M: 28.5% |
| 253J-Lung | G0-G1: 52.3% | G0-G1: 49.5% |
| | S: 35.2% | S: 25.6% |
| | G2-M: 12.5% | G2-M: 24.9% |

sHA2. 75 Causes G2-M Arrest

To confirm the effect of sHA2.75 on the G2-M phase of the cell cycle, the levels of G2-M phase check point proteins or regulators in tumor cells that were treated with sHA2.75 were determined. 253J-Lung cells were plated in culture medium at a density of 15,000 cells/plate. The cells were treated with sHA2.75 (0, 5, 10, or 20 μg/mL) for 96 hours. Following incubation, the cells were solubilized in Laemmli gel loading buffer at a concentration of 10,000 cells/10 μL. The samples were subjected to immunoblot analyses. For a loading control, the blots were probed with an anti-actin antibody (Lokeshwar et al., 2005a, 2005b). The blots were developed using a chemiluminescence kit (Amersham).

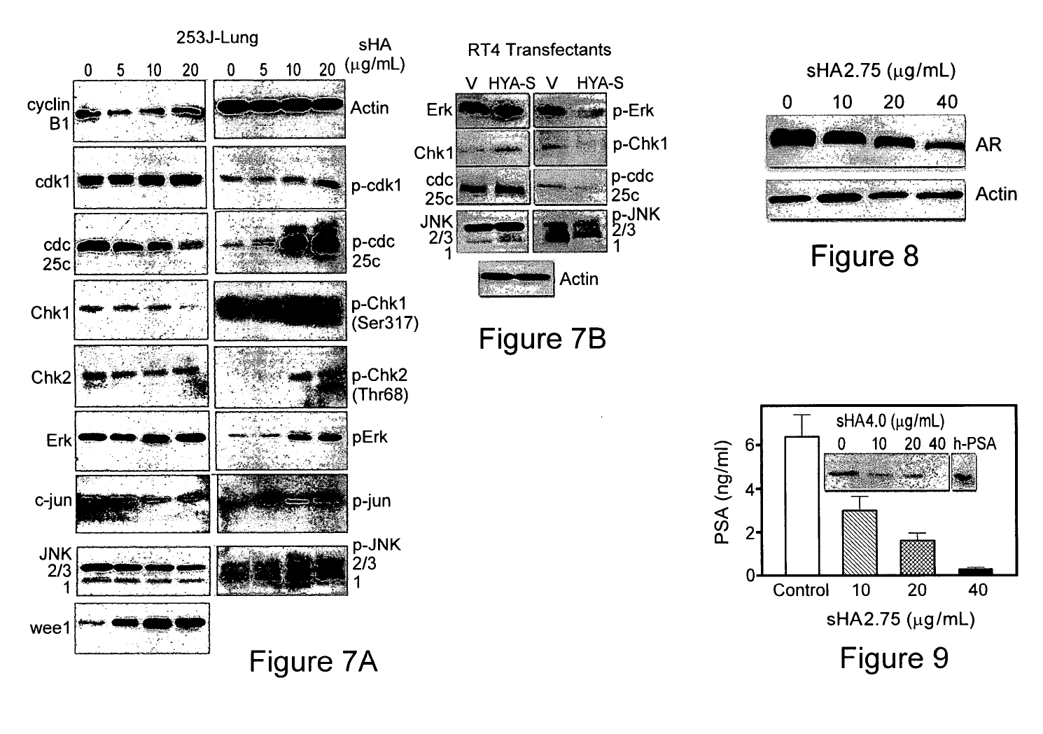

G2 to M transition requires cyclin B1/cdk1 kinase activity. Cdk1 is inactivated when phosphorylated by Myt1 and wee1 kinases. Cdc25c dephosphorylates cdk1. Upon phosphorylation by Chk1 and Chk2, however, cdc25c binds to protein 14-3-3, sequesters in the cytoplasm, and then is degraded by the proteosome mediated pathway. Chk1 and Chk2 are activated when phosphorylated by ATR and ATM, respectively. Activated Erk1/2 complexes with ATR (and possibly ATM) and induces their kinase activity. This pathway is responsible for G2-M arrest induced by several cytotoxic agents, radiation, and BRAC1. JNK activation also induces G2-M arrest by generating reactive oxygen species and/or stabilization of wee1 expression. Immunoblot analyses of 253J-Lung cells treated with sHA2.75 for 48 hours show increased p-cdc25c, p-cdk1, p-Chk1, p-Chk2, p-Erk1/2 p-JNK(1 and 2/3), p-jun, and wee1 levels but decreased cdc25c levels (FIG. 7A). Conversely, HYAL1 expression in RT4 cells by stable transfection (HYA-S) decreased p-Erk1/2, p-Chk1, p-cdc25c, and p-JNK levels (FIG. 7B) and caused a 3-fold decrease in % of cells in G2-M. Since Erk1/2 and JNK are activated by pericellular HA-CD44/RHAMM interaction, HYAL1 inhibition may cause their sustained activation and thereby result in G2-M arrest.

sHA2.75 Treatment Down Regulates AR Protein Levels

In hormone refractory CaP, tumor cells become functionally independent of androgen, however, AR signaling still plays an important role in their growth and survival (Isaacs et al., 2002; Chatterjee, 2003). Therefore, one of the mechanisms to control hormone refractory CaP would be to abrogate AR expression. Since sHA2.75 inhibited the growth of both androgen responsive LNCaP and androgen independent cells (DU145, MAT LyLu), it was hypothesized that it may down regulate AR. LNCaP cells were treated with sHA2.75 (0, 10, 20, or 40 μg/mL) for 24 hours in growth medium. Following incubation, the cell lysates were subjected to immunoblotting using an anti-AR IgG (Lab Vision). As shown in FIG. 8, AR levels decrease in a dose-dependent manner following sHA2.75 treatment: a greater than 50% decrease in AR levels is observed at 20 μg/mL of sHA2.75.

sHA2.75 Treatment Inhibits PSA Production

Since AR regulates PSA expression, whether sHA2.75 treatment down regulates PSA levels in LNCaP cells was investigated. They were treated by adding sHA2.75 to culture medium for 48 hours. Following incubation, the cell-conditioned media (CM) were assayed using a PSA ELISA kit (Research Diagnostic; Concord, Mass.) (Kizu et al., 2004). As shown in FIG. 9, sHA2.75 treatment decreased PSA levels in LNCaP CM in a dose-dependent manner; at 40 μg/mL concentration, a greater than 90% reduction in PSA levels was observed. The ELISA results were confirmed by PSA immunoblot analysis of cell lysates using an anti-human PSA IgG (FIG. 9 inset): sHA2.75 treatment causes a dose-dependent decrease in PSA protein levels. These results demonstrate that sHA2.75 belongs to a new class of compounds that down regulates both AR protein levels and AR signaling.

sHA2.75 Treatment Has Minimal Toxicity and Anti-Tumor Activity

The toxicity (if any) of sHA2.75 was evaluated in athymic mice. Athymic mice (five per group) were injected intraperitoneally with phosphate-buffered saline

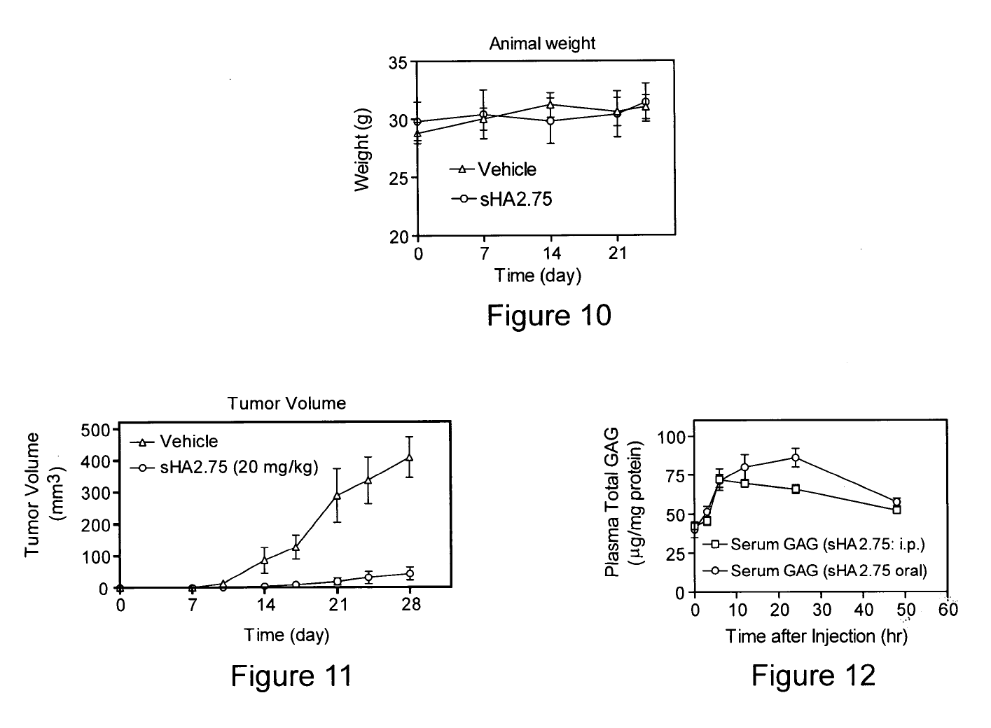

(PBS, vehicle), 10 mg/kg, 20 mg/kg, and 40 mg/kg twice weekly with sHA2.75 for 35 days. As shown in FIG. 10, no significant difference in the weights of mice was observed in the treated and untreated groups during the treatment period. No other signs of distress (lack of grooming, malaise, etc.) were observed and at necropsy no gross or histological changes were observed in kidney, lung, and liver.

To examine the effect of sHA2.75 on tumor growth, athymic mice (five per group) were injected subcutaneously with an aggressive, metastatic bladder cancer line 253J-Lung (2×10⁶ cells/site, one site/mouse). Mice were injected intraperitoneally twice weekly with 20 mg/kg sHA2.75 for 28 days. As shown in FIG. 11, 253J-Lung tumors in the untreated control group became palpable in seven days but there was a three-fold delay in the sHA-treated group (sHA2.75; 20 mg/kg). At necropsy, the tumor weight in the sHA2.75 20 mg/kg (47.5±21.2 mg) group was 70% less than that in the untreated group (141.8±44.4 mg), respectively. These results show that sHA2.75 has anti-tumor activity without any overt toxicity.

sHA2.75 is Stable in Circulation Following Parenteral or Enteral Administration

Plasma levels of sHA2.75 were determined in animals after administering the medicament by intraperitoneal injection or oral gavage. Athymic mice (8 weeks of age; 30 g weight) were either treated by intraperitoneal injection or oral gavage with 1.2 mg of sHA2.75 in PBS. Mice were euthanized at the beginning (0), 3 hours, 6 hours, 12 hours, 24 hours or 48 hours (two animals at each time) and blood was collected at necropsy. sHA2.75 concentration in plasma was measured using the carbazole assay, which measures total glycosaminoglycan concentration (Bitter & Muir, 1962). This assay was previously used to measure urinary GAG levels (Wei et al., 2000; Lokeshwar et al., 2005b).

For the carbazole assay, 50 μL aliquots of 1:1 diluted mouse sera were mixed with a sulfuric acid reagent and then a carbazole reagent, and heated at 100° C. for 25 min. Color development was measured at 515 nm. As shown in FIG. 12, when sHA2.75 was administered by intraperitoneal injection, plasma GAG concentration (pg/mg plasma protein) increased from basal level (42.2±2.3 μg/mg) over time and reached a peak at 12 hours (72.0±6.9 μg/mg). After 48 hours, plasma concentration of GAG was about 15% of the peak plasma concentration. When sHA2.75 was administered by oral gavage, plasma concentration of GAG again reached a peak at 24 hours. After 48 hours, they were 20% of the peak plasma concentration. This shows that sHA2.75 can be used as a medicament because it is fairly stable in circulation when administered by either intraperitoneal injection or oral gavage.

### REFERENCES

Barbucci et al. (1995) “Modification of hyaluronic acid by insertion of sulphate groups to obtain a heparin-like molecule. Part 1 Characterisation and behaviour in aqueous solution towards H⁺ and Ca²⁺ ions” Gazz. Chim. Ital. 125:169-180.
Bitter & Muir (1962) “A modified uronic acid carbazole reaction” Anal. Biochem. 4:330-335.
Chatterjee (2003) “The role of the androgen receptor in the development of prostatic hyperplasia and prostate cancer” Mol. Cell. Biochem. 253:89-101.
Isaacs et al. (2002) “Focus on prostate cancer” Cancer Cell 2:113-116.
Isoyama et al. (2006) “Differential selectivity of hyaluronidase inhibitors towards acidic and basic hyaluronidases” Glycobiology 16:11-21.
Kizu et al. (2004) “A new luciferase reporter gene assay for the detection of androgenic and antiandrogenic effects based on a human prostate specific antigen promoter and PC3/AR human prostate cancer cells” Anal. Sci. 20:55-59.
Lokeshwar et al. (1996) “Association of elevated levels of hyaluronidase, a matrix-degrading enzyme, with prostate cancer progression” Cancer Res. 56:651-657.
Lokeshwar et al. (1999) “Identification of bladder tumor-derived hyaluronidase: Its similarity to HYAL1” Cancer Res. 59:4464-4470.
Lokeshwar et al. (2000) “Urinary hyaluronic acid and hyaluronidase: Markers for bladder cancer detection and evaluation of grade” J. Urol. 163:348-356.
Lokeshwar et al. (2001) “Stromal and epithelial expression of tumor markers hyaluronic acid and HYAL1 hyaluronidase in prostate cancer” J. Biol. Chem. 276:11922-11932.
Lokeshwar et al. (2005a) “HYAL1 hyaluronidase: A molecular determinant of bladder tumor growth and invasion” Cancer Res. 65:2243-2250.
Lokeshwar et al. (2005b) “Urinary uronate and sulfated glycosaminoglycan levels: Markers for interstitial cystitis severity” J. Urol. 174:344-349.
Lokeshwar et al. (2005c) “HYAL1 hyaluronidase in prostate cancer: A tumor promoter and suppressor” Cancer Res. 65:7782-7789.
McLean et al. (2005) “The role of focal-adhesion kinase in cancer—a new therapeutic opportunity” Nat. Rev. Cancer 5:505-515.
Mitra et al. (2005) “Focal adhesion kinase: In command and control of cell motility” Nat. Rev. Mol. Cell. Biol. 6:56-68.
Stern & Stern (1992) “An ELISA-like assay for hyaluronidase and hyaluronidase inhibitors” Matrix 12:397-403.
Wei et al. (2000) “The association of elevated urinary total to sulfated glycosaminoglycan ratio and high molecular mass hyaluronic acid with interstitial cystitis” J. Urol. 163:1577-1583.

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of using an inhibitor of hyaluronidase, the method comprising administering an effective amount of the inhibitor of hyaluronidase to a human patient with cancer.

2. The method according to claim 1, wherein the hyaluronidase is encoded by a human HYA1 gene.

3. The method according to claim 1, wherein the cancer is carcinoma of the bladder or prostate gland.

4. The method according to claim 1, wherein the cancer is a solid tumor.

5. The method according to claim 1, wherein cell proliferation of the cancer is inhibited.

6. The method according to claim 1, wherein cell cycle progression of the cancer is blocked at G2-M phase.

7. The method according to claim 1, wherein angiogenesis of the cancer or neovascularization is reduced.

8. The method according to claim 1, wherein metastasis of the cancer is reduced.

9. The method according to claim 1, wherein the inhibitor is comprised of from 1 to 25,000 repeats of a disaccharide unit as illustrated in Formula I, which is optionally sulfated at position 1, 2, 3 or any combination thereof.

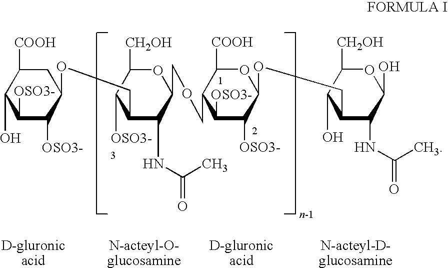

10. The method according to claim 9, wherein the inhibitor is comprised of from 3 to 30 repeats of the disaccharide unit.

11. The method according to claim 9, wherein each disaccharide unit is modified by sulfation at two or three positions.

12. The method according to claim 9, wherein at least 50% of the disaccharide units contain two sulfates and at least 25% of the disaccharide units contain three sulfates.

13. The method according to claim 9, wherein at least 25% of the disaccharide units contain two sulfates and at least 50% of the disaccharide units contain three sulfates.

14. The method according to claim 9, wherein at least 50% of the disaccharide units contain two sulfates and at least 50% of the disaccharide units contain three sulfates.

15. The method according to claim 1 further comprising treating the human patient with chemotherapy, cryotherapy, hormone therapy, radiation therapy, surgery, or any combination thereof.

16. A medicament for treatment of cancer, the medicament comprising an inhibitor of hyaluronidase and a physiologically-acceptable vehicle, which is produced and stored under aseptic conditions.

17. The medicament of claim 16 further comprising an anti-cancer agent selected from the group consisting of bicalutamide, cetuximab, docetaxel, erlotinib, etoposide, estramustine, flutamide, gallium nitrate, gemcitabine, goserelin, ifosfamide, irinotecan, leuprolide, mitoxantrone, paclitaxel, prednisone, topotecan, trastuzumab, and any combination thereof.

18. A process of using an inhibitor of hyaluronidase to manufacture a medicament for treatment of cancer comprising, under aseptic conditions:
(a) producing a medicament from an inhibitor of hyaluronidase and a physiologically-acceptable vehicle and
(b) then storing the medicament.

19. The method according to claim 9 further comprising treating the human patient with chemotherapy, cryotherapy, hormone therapy, radiation therapy, surgery, or any combination thereof.
